(19) United States
(12) Patent Application Publication
(10) Pub. No.: US 2023/0127721 A1
(43) Pub. Date: Apr. 27, 2023

(54) USE OF HAEMOGLOBIN FROM ANNELIDS FOR TREATING ACUTE RESPIRATORY DISTRESS SYNDROME

(71) Applicant: HEMARINA, Morlaix (FR)

(72) Inventor: Franck ZAL, Ploujean-Morlaix (FR)

(21) Appl. No.: 17/995,038

(22) PCT Filed: Mar. 31, 2021
(86) PCT No.: PCT/EP2021/058441

§ 371 Date: Sep. 29, 2022

(30) Foreign Application Priority Data
Apr. 2, 2020 (FR) 2003311

(51) Int. Cl. A61K 35/62; A61P 11/00; A61K 9/00

(52) U.S. Cl. CPC A61K 35/62; A61P 11/00; A61K 9/0019

(57) ABSTRACT

The present invention relates to the use of a molecule selected from an Annelid globin, an Annelid globin protomer, and an Annelid extracellular haemoglobin, to treat acute respiratory distress syndrome.

The present invention relates to the use of a molecule selected from an Annelid globin, an Annelid globin protomer, and an Annelid extracellular haemoglobin, to treat acute respiratory distress syndrome.

SARS-CoV-2 (severe acute respiratory syndrome-related coronavirus 2), the virus responsible for COVID-2019 (i.e. coronavirus disease 2019), is a novel coronavirus discovered in the city of Wuhan, Hubei province, China, in December 2019. It is responsible for an epidemic that initially had its epicentre in China. Since early 2020, the epidemic has spread to many countries, including Europe, including France, and now the United States.

Coronaviruses are enveloped RNA viruses belonging to the family Coronaviridae. Although most diseases are benign in humans, they can sometimes cause more serious situations, including respiratory tract infections such as SARS-CoV (severe acute respiratory syndrome-related coronavirus), MERS-CoV (Middle East respiratory syndrome-related coronavirus) and now SARS-CoV-2.

The incubation period of SARS-CoV-2 appears to be 5 days on average (range 2-12 days), with an epidemic doubling time of 6.4-7.5 days. The period of contagiousness is not yet well defined and it is not excluded that a person can be contagious in the 24 hours preceding the symptoms.

From a clinical point of view, for the vast majority of infected patients, non-serious forms are observed, such as the presence of upper respiratory tract infections (rhinopharyngitis, coughing or odynophagia), conjunctivitis or flu-like syndrome with headache, myalgia, asthenia and sometimes diarrhoea.

However, in a smaller number of infected patients, severe forms of the disease, such as hypoxaemic pneumonia or acute respiratory distress syndrome (ARDS), are observed. In these cases, oxygen therapy is required, and the affected individuals must be hospitalised.

There is currently no treatment or vaccine for the disease.

Furthermore, ARDS is one of the most critical stages, although it is not exclusively related to COVID-19.

Indeed, acute respiratory distress syndrome (ARDS) is a very severe form of acute lung failure, resulting from a drastic alteration in capillary permeability. It is characterised by inflammation of the lung parenchyma leading to gas exchange abnormalities with a parallel release of inflammatory mediators from the lung parenchyma causing inflammation and hypoxaemia; often multi-visceral failure results.

Four elements are necessary and sufficient for the diagnosis of ARDS (according to “le syndrome de détresse respiratoire aiguë (SDRA)”, 193a, Dominique Falcon, August 2002):
1. The patient presents with acute respiratory distress (which excludes chronic diseases such as interstitial fibrosis, sarcoidosis, or decompensated respiratory failure).
2. Hypoxia is severe and resistant to oxygen therapy alone. Its extent is assessed by the ratio of arterial partial pressure of oxygen to the inspired fraction of oxygen (PaO2/FiO2), in the absence of positive tele-expiratory pressure. This ratio is less than 300 mmHg in ARDS.
Severity is assessed according to the P/F ratio:
Mild ARDS: PaO₂/FiO₂ between 200 and 300 mmHg with positive expiratory pressure (PEEP) or continuous positive airway pressure (CPAP) ventilation≥5 cm H₂O,
Moderate ARDS: PaO₂/FiO₂ between 100 and 200 mmHg with PEEP≥5 cm H₂O,
Severe ARDS: PaO₂/FiO₂≤100 mmHg with PEEP≥5 cm H₂O.
3. The frontal chest X-ray shows diffuse, bilateral, unsystematised alveolar images consistent with pulmonary oedema. This excludes hypoxia after pulmonary embolism or single lung disease (pneumopathy).
4. This pulmonary oedema should not be the consequence of left heart failure.
The treatment of ARDS, both etiological and symptomatic, allows survival in only half of the cases of so-called “severe” ARDS. Its prognosis is therefore still very poor and it can leave significant after-effects.

There is therefore a need for effective and rapid treatment of ARDS, in particular ARDS caused by COVID-19.

The present invention makes it possible to meet these expectations.

Surprisingly, the Applicant has found that administration of at least one molecule selected from Annelid globin, Annelid globin protomer, and Annelid extracellular haemoglobin, in patients with ARDS, provides oxygen directly to the tissues. This administration of the molecule to the ARDS patient restores the oxygen-carrying capacity of the blood.

Patients with ARDS according to the invention are typically human patients. “Oxygen-carrying capacity of the blood” refers to the total amount of O2 taken up by the blood under saturated conditions. Thus, in humans, with a normal haemoglobin level of 14 to 15 g/dl, the carrying capacity of one decilitre of blood is about 20 ml of oxygen (i.e. an oxygen capacity of 20 ml O2/dL of blood).

The present invention relates to the use of a molecule selected from an Annelid globin, an Annelid globin protomer, and an Annelid extracellular haemoglobin, to treat acute respiratory distress syndrome (ARDS).

The use of a molecule selected from an Annelid globin, an Annelid globin protomer, and an Annelid extracellular haemoglobin according to the invention makes it possible to increase the amount of oxygen in the blood, and thus to improve its oxygen-carrying capacity and/or its saturation.

Indeed, the administration of a molecule selected from an Annelid globin, an Annelid globin protomer, and an Annelid extracellular haemoglobin, to treat ARDS according to the invention, makes it possible to supply oxygen in situ to the patient's tissues. Thus, PaO2 and/or saturation is increased. Furthermore, the administration of said molecule is typically into the patient's bloodstream, so it is circulating; it can thus regenerate into oxygen in the patient's lungs, and provide continuous oxygen to the tissues. Typically, the effect lasts a few days, for example at least 2 or 3 days.

Preferably, the ARDS patient of interest for the invention is a hypoxic human patient.

Preferably, the present invention relates to the use of a molecule selected from an Annelid globin, an Annelid globin protomer and an Annelid extracellular haemoglobin, for treating acute respiratory distress syndrome (ARDS) in hypoxic human patients.

Preferably, the ARDS patient of interest for the invention is a human patient with a loss of blood oxygen-carrying capacity of at least 3 ml O2/dL of blood, preferably at least 4 ml O2/dL of blood, more preferably at least 5 ml O2/dL of blood.

Typically, the oxygen-carrying capacity of blood is measured in the usual way, either indirectly by measuring the concentration of red blood cells (haematocrit) or directly by determining the concentration of haemoglobin.

Preferably, the present invention relates to the use of a molecule selected from an Annelid globin, an Annelid globin protomer, and an Annelid extracellular haemoglobin, for treating ARDS in human patients with a loss of blood oxygen-carrying capacity of at least 3 ml O2/dL of blood, preferably at least 4 ml O2/dL of blood, more preferably at least 5 ml O2/dL of blood.

Preferably, the ARDS patient of interest for the invention is a human patient with a low oxygen saturation, i.e. less than 85%, preferably less than 80%.

Preferably, the present invention relates to the use of a molecule selected from an Annelid globin, an Annelid globin protomer and an Annelid extracellular haemoglobin, for treating ARDS in human patients with low oxygen saturation, i.e. below 85%, preferably below 80%.

In one embodiment, ARDS is caused by infection with the SARS-Cov-2 coronavirus (i.e. ARDS caused by COVID-19).

Preferably, the present invention relates to the use of a molecule selected from an Annelid globin, an Annelid globin protomer, and an Annelid extracellular haemoglobin, for treating ARDS in human patients infected with the SARS-Cov-2 coronavirus and/or with COVID-19.

The molecule according to the invention is selected from an Annelid globin, an Annelid globin protomer, and an Annelid extracellular haemoglobin.

This molecule is an oxygen carrier. “Oxygen carrier” means a molecule capable of reversibly transporting oxygen from the environment to target cells, tissues or organs.

Annelid extracellular haemoglobin is present in all three classes of Annelids: Polychaetes, Oligochaetes and Hirudinea (Achaetes). It is called extracellular haemoglobin because it is naturally not contained in a cell, and can therefore circulate freely in the bloodstream without chemical modification to stabilise it or make it functional.

Annelid extracellular haemoglobin is a giant biopolymer with a molecular weight of between 2,000 and 4,000 kDa, consisting of about 200 polypeptide chains of between 4 and 12 different types that are generally grouped into two categories.

The first category, comprising 144 to 192 elements, groups together the so-called “functional” polypeptide chains that carry a heme-type active site and are capable of reversibly binding oxygen; these are globin-type chains (eight types in total for the haemoglobin of Arenicola marina: a1, a2, b1, b2, b3, c, d1 and d2), whose masses are between 15 and 18 kDa. They are very similar to the α and β chains of vertebrates.

The second category, with 36 to 42 elements, groups together the polypeptide chains known as “structure” or “linkers” with little or no active site but which allow the assembly of sub-units called twelfths or protomers. There are two types of linkers, L1 and L2.

Each haemoglobin molecule is made up of two overlapping hexagons, known as the hexagonal bilayer, and each hexagon is formed by the assembly of six subunits (dodecamer or protomer) in the shape of a teardrop. The native molecule is made up of twelve of these subunits (dodecamer or protomer). Each subunit has a molecular weight of approximately 250 kDa, and is the functional unit of the native molecule.

Preferably, the extracellular haemoglobin of Annelids is selected from the extracellular haemoglobins of Polychaetes Annelids and the extracellular haemoglobins of Oligochaetes Annelids. Preferably, the extracellular haemoglobin of Annelids is selected from extracellular haemoglobins of the family Lumbricidae, extracellular haemoglobins of the family Arenicolidae and extracellular haemoglobins of the family Nereididae. Even more preferably, the extracellular haemoglobin of Annelids is selected from extracellular haemoglobin of Lumbricus terrestris, extracellular haemoglobin of Arenicola sp and extracellular haemoglobin of Nereis sp. More preferably according to the invention, the extracellular haemoglobin of Arenicola marina or Nereis virens, more preferably the extracellular haemoglobin of Arenicola marina. The arenicola Arenicola marina is a polychaete annelid worm that lives mainly in the sand.

According to the invention, the globin protomer of Annelid extracellular haemoglobin constitutes the functional unit of native haemoglobin, as described above.

Finally, the globin chain of the Annelid extracellular haemoglobin may in particular be selected from the Ax and/or Bx type globin chains of Annelid extracellular haemoglobin.

Annelid extracellular haemoglobin, its globin protomers and/or globins do not require a cofactor to function, unlike mammalian, especially human, haemoglobin. Finally, since

Annelid extracellular haemoglobin, its globin protomers and/or globins do not have blood typing, they avoid any problems of immunological or allergic reactions. Annelid extracellular haemoglobin, its globin protomers and/or its globins exhibit intrinsic superoxide dismutase (SOD) activity. Therefore, this intrinsic antioxidant activity does not require any antioxidants to function, unlike the use of mammalian haemoglobin where the antioxidant molecules are contained within the red blood cell and are not bound to the haemoglobin.

Annelid extracellular haemoglobin, its globin protomers and/or its globins may be native or recombinant.

Preferably, the extracellular haemoglobin is that of Arenicola marina.

Preferably, the molecule selected from an Annelid globin, an Annelid globin protomer, and an Annelid extracellular haemoglobin according to the invention, is formulated in a composition comprising a buffer solution.

The composition, called the composition according to the invention, then comprises the molecule according to the invention, dissolved in the buffer solution.

Preferably, the molecule selected from an Annelid globin, an Annelid globin protomer and an Annelid extracellular haemoglobin according to the invention, is present in the composition according to the invention in a concentration of between 1 and 200 g/L, preferably between 5 and 100 g/L, more preferably between 10 and 80 g/L.

The buffer solution creates a suitable salt environment for haemoglobin, its protomers, and its globins, and thus allows the maintenance of the quaternary structure, and thus the functionality of this molecule.

The buffer solution according to the invention is preferably an aqueous solution comprising salts, preferably chloride, sodium, calcium, magnesium and potassium ions, and gives the composition according to the invention a pH of between 5 and 9, preferably between 5.5 and 8.5, most preferably between 6.5 and 7.6. Its formulation is similar to that of a physiologically injectable liquid. Under these conditions, Annelid extracellular haemoglobin, its globin protomers, and its globins remain functional.

In this description, pH is understood to be at room temperature (25° C.), unless otherwise stated. Preferably, the buffer solution is an aqueous solution comprising sodium chloride, calcium chloride, magnesium chloride, potassium chloride, as well as sodium gluconate and sodium acetate, and has a pH of between 6.5 and 7.6, preferably equal to 7.1±0.5, preferably about 7.35. More preferably, the buffer solution is an aqueous solution comprising 90 mM NaCl, 23 mM Na-gluconate, 2.5 mM CaCl₂, 27 mM Na-acetate, 1.5 mM MgCl₂, 5 mM KCl, and has a pH of 7.1±0.5.

Preferably, the buffer solution also includes at least one stabilising agent. This stabilising agent ensures the maintenance of the quaternary structure and thus the functionality of haemoglobin, its globins, and its protomers. The stabilising agent according to the invention is preferably selected from disaccharides, polyols, antioxidants, maltodextrins, and mixtures thereof.

Preferably, the disaccharides are selected from sucrose, trehalose and raffinose, preferably from trehalose and sucrose, and most preferably trehalose. Preferably, the polyols are selected from mannitol and sorbitol. Preferably, the antioxidant is ascorbic acid or reduced glutathione, preferably ascorbic acid.

Trehalose is also called α-D-glucopyranosyl-α-D-glucopyranoside or alpha,alpha-threhalose, or α-D-glucopyranosyl-α-D-glucopyranoside, dihydrate. It is a disaccharide composed of two glucose molecules linked together by a particularly stable α,α-1,1 (or “1,1-α-glycosidic”) bond.

Sucrose is a disaccharide formed by the condensation of a glucose molecule with a fructose molecule. Its chemical name is β-D-fructofuranosyl-(2↔1)-α-D-glucopyranoside.

Mannitol, or 1,2,3,4,5,6-hexanehexol, and sorbitol, or (2R,3S,4S,5S)-Hexane-1,2,3,4,5,6-hexol, are polyols.

Ascorbic acid is an organic acid with antioxidant properties. It may be present in D or L form. Preferably, the stabilising agent is L-ascorbic acid.

Maltodextrins are polymers of glucose units. Preferably, the maltodextrin is maltodextrin DE 7, which is a linear chain of glucoses linked by α(1↔4) osidic bonds. The length of the chain varies between 3 and 20 units. The DE (Dextrose Equivalent) is a measure of the amount of reduced sugar in the product; the dextrose being D-Glucose. The DE is a percentage of the reducing power of dextrose (which is 100) giving information on the degree of polymerisation of the maltodextrin.

Preferably, the stabilising agent is selected from trehalose, mannitol, ascorbic acid, maltodextrins, and mixtures thereof. Preferably, the stabilising agent is a mixture of trehalose, mannitol, and ascorbic acid.

Preferably, the stabilising agent is present in the composition according to the invention in a concentration between 1 and 500 g/L, preferably between 5 and 100 g/L.

More preferably, the stabilising agent is at least one disaccharide, preferably trehalose, present in a concentration between 5 and 30 g/L, preferably between 10 and 20 g/L.

More preferably, the stabilising agent is at least one polyol, preferably mannitol, present in a concentration between 1 and 30 g/L, preferably between 5 and 20 g/L.

More preferably, the stabilising agent is at least one antioxidant, preferably ascorbic acid, present in a concentration of between 1 and 20 g/L, preferably between 5 and 20 g/L.

More preferably, the stabilising agent is at least one maltodextrin, present in a concentration between 1 and 100 g/L, preferably between 5 and 50 g/L.

According to a first embodiment, the molecule selected from an Annelid globin, an Annelid globin protomer, and an Annelid extracellular haemoglobin is formulated in a composition in liquid form.

According to a first embodiment, the molecule selected from an Annelid globin, an Annelid globin protomer, and an Annelid extracellular haemoglobin is formulated in a composition in powder form.

Preferably, the composition according to the invention, comprising the molecule selected from an Annelid globin, an Annelid globin protomer and an Annelid extracellularhaemoglobin, and a buffer solution, is in powder form.

This powder can be obtained by drying, by atomisation, or by freeze-drying.

In the case of freeze-drying, the powder can be obtained in the following steps:
i) the mixture of the molecule selected from an Annelid globin, an Annelid globin protomer, and an Annelid extracellular haemoglobin according to the invention, a buffer solution, and at least one stabilising agent selected from disaccharides, polyols, antioxidants, maltodextrins and mixtures thereof,
ii) freezing the mixture obtained in i) at a temperature of between −20° C. and −100° C. for a time of at least 24 hours, preferably at least 48 hours;
iii) sublimating the frozen mixture obtained in (ii) for at least 2 hours, under vacuum;
iv) final drying of the mixture obtained in iii), until a powder is obtained.

The mixing in step i) can be done by vortexing.

The freeze-drying cycle, i.e. steps ii) to iv), consists of three steps:
freezing (step ii):

This first phase consists of freezing the solution in such a way that the water contained is transformed into ice.

Preferably, the freezing in step ii) of the method according to the invention is carried out at a temperature between −20° C. and −90° C. for at least 24 hours, preferably at least 48 hours. Preferably, freezing is carried out at about −80° C. for at least 24 hours, preferably at least 48 hours.
primary drying or sublimation (stage iii):

The sublimation step allows the ice present in the frozen solution to pass from the solid state to the gaseous state, without any intermediate step. The frozen solution is dried out by means of a vacuum; the ice then becomes steam.

Sublimation is done using a high vacuum pump, a mechanical pump or a cryo-pump.

Preferably, the sublimation in step iii) is carried out for at least 4 hours.
secondary drying or final drying (step iv):

When the ice is completely sublimated, the secondary drying phase can begin. It allows the water molecules trapped on the surface of dried products to be extracted by desorption.

At the end of the freeze-drying process, the resulting freeze-dried product contains between 1 and 5% by weight of water.

Preferably, when the composition according to the invention is in powder form, it can be completely redissolved in a hydrophilic liquid (diluent), without insoluble residues. The powder can be stored in glass or plastic bottles or flasks, preferably glass.

Preferably, the composition according to the invention is in liquid form or in powder form in single-dose vials.

The molecule selected from Annelid globin, Annelid globin protomer, and Annelid extracellular haemoglobin, thus obtained, is easy to transport and store, but also easy to reconstitute and ready for use.

Preferably, the molecule selected from an Annelid globin, an Annelid globin protomer, and an Annelid extracellular haemoglobin according to the invention, or the composition according to the invention, is administered directly to the patient with ARDS.

Preferably, the molecule selected from an Annelid globin, an Annelid globin protomer, and an Annelid extracellular haemoglobin according to the invention, or the composition according to the invention, is administered enterally or parenterally.

Preferably, the molecule selected from an Annelid globin, an Annelid globin protomer and an Annelid extracellular haemoglobin according to the invention, or the composition according to the invention, is administered by injection, preferably intramuscularly, subcutaneously, intra-arterially or intravenously, more preferably intravenously.

Preferably, the molecule selected from an Annelid globin, an Annelid globin protomer, and an Annelid extracellular haemoglobin according to the invention, or the composition according to the invention, is administered by injection, in particular intravenously. Typically, in this case, said molecule is administered at a dose between 0.5 g/L and 70 g/L of blood, preferably between 0.7 g/L and 50 g/L, more preferably between 0.8 /L and 10 g/L.

### EXAMPLE

### Combating Hypoxaemia in COVID-19 Patients Using Extracellular Haemoglobin From Arenicola marina

The hypothesis is that intravenous injection of a composition comprising Arenicola marina extracellular haemoglobin (HEMO2Life®) in ARDS due to COVID-19 would improve oxygen transport to the tissues, and that this could prevent progression to multi-organ failure if hypoxaemia persists or worsens.

This molecule has been administered to humans for transplantation, as an additive in a preservative solution, but never directly by intravenous route.

The use of extracellular haemoglobin from Arenicola marina is also interesting because of its antioxidant effect, which prevents the cytokine storm induced by SARS-CoV-2. Indeed, the extracellular haemoglobin of Arenicola marina has a superoxide dismutase activity that can solve this problem.

Extracellular haemoglobin from Arenicola marina can improve tissue oxygenation without altering ventilation for COVID-19 patients. This extracellular haemoglobin has an oxygen binding capacity 40 times greater than vertebrate haemoglobin. Moreover, the size of this molecule is 250 times smaller than a human red blood cell, which allows it to diffuse into all areas of the microcirculation without diffusing out of the vascular sector. This molecule is composed of 156 globin chains and 42 linker chains with a molecular weight of 3.6 MDa. The quaternary structure of this molecule is a hexagonal bilayer with a dimension of 25 nm (front view) and 15 nm (side view). Each globin chain has a heme group similar to human, and the linker chains have an antioxidant property due to a copper and zinc-based superoxide dismutase (SOD)-like activity. Thus, this haemoglobin can carry up to 156 molecules of O2. Oxygen is released against a gradient in the absence of an allosteric effector, supplying the environment with the right amount of O2. It is active over a wide temperature range (4° C. to 37° C.). This molecule has no immunogenic or allergenic effect. It has an oxygen affinity (p50) of 7.5 mm Hg (i.e. similar to that of haemoglobin A (HbA) within the red blood cell), has a cooperativity of 2.5 and does not require a cofactor to release oxygen. In addition, the p50 of myoglobin is 2.6 mm Hg, which is less than 7.5 mm Hg. The release of O2 simply takes place in an oxygen gradient: when the pO2 is lower than the p50, O2 is passively released to the tissues, and consumed by the cells or tissues, avoiding oxidative damage. There is no interaction between the extracellular haemoglobin of Arenicola marina and haemopexin, an important plasma protein in the clearance of haemoglobin.

SARS-CoV-2 is an enveloped single-stranded RNA virus that replicates in the nuclei of target cells. The DNA in the nucleus of red blood cells is therefore probably one of the targets of the virus, and this explains the leukoerythroblastic reaction described in a patient with COVID-19. To date, few studies have provided data on the use of blood in patients with COVID-19. It has been argued that patients hospitalised with COVID-19 required fewer blood transfusions than other hospitalised patients. Data from Italy showed that 39% of patients required transfusion (median hospital stay 15 days) mainly for anaemia (without bleeding), with very few patients requiring platelets or plasma.

The extracellular haemoglobin of Arenicola marina is not contained in the cell nucleus and therefore cannot be a target for the virus as SARS-CoV-2 will not recognise this non-red blood cell oxygen carrier. It seems that the virus must attach to the red cell with more affinity for blood group AB, which will not be possible with extracellular haemoglobin. Therefore, this molecule seems well suited to deliver oxygen and avoid hypoxia responsible for dyspnoea, while avoiding being targeted by the virus.

Another reason to use extracellular haemoglobin from Arenicola marina is related to its oxidative stress reducing properties. SARS-CoV-2 acts on the angiotensin converting enzyme 2 (ACE2) receptor. By binding to the ACE2 receptor, the virus inhibits the conversion of angiotensin II to angiotensin 1,7. The latter is fundamental to NADPH oxidase: this enzyme catalyses the oxidation reaction of NADPH by oxygen, which creates reactive oxygen species (ROS), which are toxic and generate endothelial dysfunction. The extracellular haemoglobin of Arenicola marina, through its SOD-like properties, can reverse this phenomenon by changing O2∘ to O2 or H2O2.

The extracellular haemoglobin of Arenicola marina also has an action on iron, and may potentially stimulate catalase. COVID-19 causes hypoxia due to anaemia, coagulopathy, thrombosis and multiple organ failure. Lung damage observed on radiographic scans may be caused by the release of oxidative iron from heme groups, overwhelming natural defences against pulmonary oxidative stress; elevated ferritin levels are also found in non-surviving COVID-19 patients compared to surviving patients. The function of catalase is to detoxify free circulating heme, which can cause severe inflammation. Indeed, when iron ions are depleted of haemoglobin, intubation to ventilate is useless as it does not treat the cause of the disease, and iron in free form could be responsible for the cytokine storm due to its very high pro-oxidant activity. The fact that patients return for re-hospitalisation days or weeks after recovery and suffer delayed post-hypoxic leukoencephalopathy reinforces the fact that COVID-19 patients suffer from hypoxia despite no signs of respiratory fatigue or exhaustion.

Tissue hypoxia, although rarely assessed in the literature, could be an interesting complementary assessment measure.

Red blood cells carry oxygen from the lungs to all the organs and the rest of the body through haemoglobin. This protein consists of four “hemes”, which contain a special type of iron ion, which is usually quite toxic in its free form, and enclosed in a porphyrin at its centre. In case of COVID-19 infection, the lungs are overwhelmed with oxidative stress, the organs need a lot of O2 and the liver does its best to eliminate and store iron. However, this organ also needs O2, and releases an enzyme called alanine aminotransferase. The patient's immune system cannot fight the virus until the oxygen saturation is too low, and the organs begin to shut down. To avoid this, a maximum supply of oxygen is necessary. The extracellular haemoglobin of Arenicola marina can provide this O2.

It may also play a role in the treatment of microthrombosis in SARS-CoV-2 infections. Histological analyses of skin and lung patients have shown microvascular lesions and thrombosis associated with severe forms of COVID-19, and a retrospective study of 183 patients shows abnormal coagulation results, in particular high levels of D-dimer and fibrin degradation products in COVID-19 deaths. This microthrombosis is due to a cascade of events causing the destruction of the vascular endothelium by ROS. This microphenomenon of thrombosis can lead to acute respiratory failure and systemic coagulopathy, which are critical to the morbidity and mortality of SARS-CoV-2 infection. As the extracellular haemoglobin of Arenicola marina is 250 times smaller than red blood cells and extracellular, it can cross the thrombus generated by SARS-CoV-2. This hypothesis is also supported by the fact that, in a rat model affected by head trauma, and therefore highly susceptible to intravascular micro-thrombosis, extracellular haemoglobin from Arenicola marina could rapidly reduce acute cerebral hypoxia tissue, avoiding the classical reduction in vessel size without inducing vasoconstriction itself.

Indeed, the extracellular haemoglobin of Arenicola marina has no vasoconstrictor effect compared to other first or second generation oxygen carriers.

The extracellular haemoglobin of Arenicola marina is well tolerated and does not induce toxicity. It is pyrogen-free, non-mutagenic, non-cytotoxic and non-irritating. When administered intravenously to hamsters and rats, it showed no acute effects on heart rate and blood pressure, and did not induce microvascular vasoconstriction.

In another study, fluorescently labelled Arenicola marina extracellular haemoglobin was administered to mice (60 mg/kg, 600 mg/kg, 1200 mg/kg) and was found to be safe, the animals showed no abnormal clinical signs and the half-life of the product was 2.5 days.

The extracellular haemoglobin of Arenicola marina was evaluated in the human kidney in the OXYOP study (NCT02652520). This study, the first in humans, demonstrated that the addition of extracellular haemoglobin from Arenicola marina to a kidney transplant preservation solution is safe. Although this study was not designed to show the superiority of Arenicola marina extracellular haemoglobin, analysis of the secondary efficacy endpoints shows significantly less delayed graft function recovery and better renal function in recipients of kidneys preserved with this haemoglobin. This study calls for the use of extracellular haemoglobin from Arenicola marina in organ preservation. This also shows the relevance of using this haemoglobin in diseases related to ischaemia-reperfusion injury and hypoxia.

Some oxygen transporters have been studied and shown to be effective in a preclinical model of ARDS.

For example, in 2004, Henderson et al. evaluated whether a cross-linked and polymerised bovine haemoglobin (HBOC-201 from Biopure) is an alternative to donor blood for extracorporeal oxygenation in a pig model of ARDS. HBOC-201 appears to be an effective alternative for extracorporeal membrane oxygenation, offering the advantages of rapid availability and reduced exposure to donor blood cells.

Extracellular haemoglobin from Arenicola marina has not yet been studied in preclinical studies for this condition, but superior efficacy can be expected, as it did not induce vasoconstriction as demonstrated in comparison with first- and second-generation oxygen carriers. Deeply hypoxaemic patients admitted to the ICU under COVID-19 may be a population that could benefit from intravenous administration of Arenicola marina extracellular haemoglobin.

Given its oxygen-carrying and oxidative stress reduction properties, extracellular haemoglobin from Arenicola marina may be effective in combating hypoxia and oxidative stress caused by SARS-CoV-2.

It is estimated that the intake of 5 g of this haemoglobin for a 70 kg subject (70 mg/kg), whose blood volume is estimated to be 5 L, represents an increase in arterial O2 content of 1 ml of O2 per 100 mL of blood (or 5% of the “physiological” oxygen content of arterial blood (CaO2) or 7% if the partial pressure of oxygen (PAO2) is 80 mmHg). Administration may be started with a “test dose” of 10 mg to check for anaphylaxis. Then each 1 g dose can be administered intravenously. An assessment of tolerance can be made after each dose, looking for rashes, bronchospasm, hypotension or tachycardia during the next 5 minutes before proceeding to the next dose. If the administration of 70 mg/kg haemoglobin (i.e. 1.4 ml/kg) does not significantly improve tissue oxygenation parameters, and if the dose is well tolerated, then 70 mg/kg haemoglobin can be administered for a total of 140 mg/kg, which corresponds to a 10% increase in CaO2. As this haemoglobin has a 40-fold higher carrying capacity than HbA, it could increase the arterial O2 content in a situation where the pulmonary exchanger is no longer functional, whereas O2 binding and release occurs passively in a simple O2 gradient in the absence of an allosteric effector.

Extracellular haemoglobin from Arenicola marina could improve survival of COVID-19 patients, avoid tracheal intubation, shorten oxygen supplementation and treat more patients when ventilators are not available.

1. A method for treating acute respiratory distress syndrome in patients with a loss of blood oxygen capacity of at least 3 ml O2/dL of blood, comprising administering to said patients at least one molecule selected from an Annelid globin, an Annelid globin protomer and an Annelid extracellular haemoglobin.

2. The method according to claim 1, characterised in that the molecule is selected from extracellular haemoglobins of the family Lumbricidae, extracellular haemoglobins of the family Arenicolidae and extracellular haemoglobins of the family Nereididae, preferably from the extracellular haemoglobin of Lumbricus terrestris, the extracellular haemoglobin of Arenicola sp and the extracellular haemoglobin of Nereis sp, more preferably from the extracellular haemoglobin of Arenicola marina and Nereis virens.

3. The method according to claim 1, characterised in that the molecule is the extracellular haemoglobin of Arenicola marina.

4. The method according to claim 1, characterised in that the acute respiratory distress syndrome is caused by infection with the coronavirus SARS-Cov-2.

5. The method according to claim 1, characterized in that the acute respiratory distress syndrome is present in patients with a loss of blood oxygen-carrying capacity of at least 4 ml O2/dL of blood, preferably at least 5 ml O2/dL of blood.

6. The method according to claim 1, characterised in that the acute respiratory distress syndrome is present in patients with low oxygen saturation, i.e. less than 85%, preferably less than 80%.

7. The method according to claim 1, characterised in that the molecule is formulated in a composition comprising a buffer solution.

8. The method according to claim 7, characterized in that the buffer solution is an aqueous solution comprising salts, preferably chloride, sodium, calcium, magnesium and potassium ions, and gives the composition a pH between 5 and 9, preferably between 5.5 and 8.5, preferably between 6.5 and 7.6, and preferably also comprises at least one stabilizing agent, preferably selected from disaccharides, polyols, antioxidants, maltodextrins and mixtures thereof.

9. The method according to claim 7, characterized in that the molecule is present in the composition in a concentration of between 1 and 200 g/L, preferably between 5 and 100 g/L, more preferably between 10 and 80 g/L.

10. The method according to claim 1, characterised in that the molecule is formulated in a composition in powder form.

11. The method according to claim 1, characterised in that the molecule is in a form adapted to be administered enteral or parenterally, preferably by injection, preferably intramuscularly, subcutaneously, intra-arterially or intravenously, more preferably intravenously.
